(19) United States
(12) Patent Application Publication
(10) Pub. No.: US 2014/0261428 A1
(43) Pub. Date: Sep. 18, 2014

(54) Respiratory Filter Device

(71) Applicant: Yu-Hu Chen, New Taipei City (TW)

(72) Inventor: Yu-Hu Chen, New Taipei City (TW)

(21) Appl. No.: 14/200,110

(22) Filed: Mar. 7, 2014

(30) Foreign Application Priority Data
Mar. 12, 2013 (TW) 102204454

(51) Int. Cl. A61M 16/10; A61M 16/00; A61M 16/06

(52) U.S. Cl. CPC A61M 16/105; A61M 16/06; A61M 16/0066
USPC 128/205.12; 128/206.12

(57) ABSTRACT

Provided is a respiratory filter device having a filter module, a connection curved holder, an adjustable curved holder, and a mask. The filter module has a housing body, and both the connection curved holder and the adjustable curved holder have a respective free end and a respective connection end opposite to each other. The connection ends of the connection curved holder and the adjustable curved holder are connected with the filter module, and the free ends face to each other and form an opening between the free ends. The filter module is connected to the connection curved holder and the adjustable curved holder to define an accommodation space adjacent to the opening. Accordingly, the respiratory filter device can be worn on a user's head or neck directly by the accommodation space, for allowing the user to do other work when wearing the respiratory filter device.

### RELATED APPLICATION

The present invention claims the priority of Taiwan Patent Application No. 102204454 filed on Mar. 12, 2013, which is incorporated by reference in the present application in its entirety.

### BACKGROUND OF THE INVENTION

1. Field of the Invention

The present invention relates to a respiratory filter device, and more particularly to a respiratory filter device convenient for users to wear on their necks or heads.

2. Description of the Prior Arts

With the increasing importance attributed to health care, masks made in various types become indispensable items for outdoor occasions. In order to effectively block the harmful substances, such as dust mite and viruses, most masks in the art are typically equipped with multiple filters to isolate the harmful substances in the ambient air from a user's nose and mouth. However, the mask covering over the nose and mouth tends to cause stuffiness during wearing, thereby reducing the user's intention to wear the mask.

Another respiratory protection assembly, including a filter apparatus, a mask and a tube connected thererwith, is developed to overcome the foregoing drawbacks. With the function of the filter apparatus, the harmful substances contained in the ambient air are filtered away in advance, ensuring that the user inhales purified air.

Nevertheless, the filter apparatus in the art cannot be directly worn on the user's body, which brings much inconvenience when the user is doing other work and has to hold the filter apparatus in hand. Further, to allow the user to carry the filter apparatus with a belt on the waist, the tube used to connect the filter apparatus with the mask has to be made longer, such that a user carrying the filter apparatus is more likely to drag the tube and thus separate the filter apparatus form the mask accidentally. The isolation effect of the respiratory protection assembly in the art is thus reduced.

### SUMMARY OF THE INVENTION

To overcome the shortcomings, an objective of the present invention is to provide a respiratory filter device to mitigate or obviate the aforementioned problems, thereby improving the convenience for wearing and use.

To achieve the objective, the present invention provides a respiratory filter device, having:

a filter module having a housing body and a filter element mounted on the housing body, the housing body having an inlet and an outlet communicating with the inlet, and the filter element mounted between the inlet and the outlet;

a connection curved holder connected with the filter module, and having a free end and a connection end connected with the housing body;

an adjustable curved holder connected with the filter module and opposite to the connection curved holder, the adjustable curved holder having a connection end connected with the housing body and a free end facing the free end of the connection curved holder with an opening between the free ends of the connection curved holder and the adjustable curved holder;

a connection tube embedded in the connection curved holder and having two opposite ends, one of the ends of the connection tube connected to the outlet of the housing body; and

a mask connected with the connection tube and having a breathing space communicating with the other end of the connection tube;

wherein the filter module is connected to the connection curved holder and the adjustable curved holder to define an accommodation space adjacent to the opening.

In accordance with the present invention, the respiratory filter device of the present invention has beneficial effects of:

(1) convenience in use:

With the configurations of the opening and the accommodation space, the respiratory filter device of the present invention can be simply mounted around a user's head or neck, and the length of the connection tube can be further shortened by wearing the respiratory filter device on a user's head or neck. Accordingly, the inconvenience of holding a conventional respiratory filter device in hand or carrying on the waist is overcome, and the respiratory filter device in accordance with the present invention is convenient for a user to do other work without accidentally dragging the connection tube when wearing the respiratory filter device

(2) stable connection between the connection tube and the filter module:

Since the connection tube is embedded in the connection curved holder connected with the housing body, the connection tube is fully protected by the connection curved holder and is securely connected with the housing body. Accordingly, the respiratory filter device has a resistance to accidental drop upon wearing and provides a good filtering effect.

Preferably, the opening between the free ends of the connection curved holder and the adjustable curved holder is smaller than the size of a user's head and neck, and is smaller than the size of the accommodation space, such that the respiratory filter device is securely mounted around a user's head or neck without accidental drop.

Preferably, the adjustable curved holder is rotatably connected with the housing body of the filter module. More preferably, the connection curved holder and the adjustable curved holder are rotatably connected with two opposite sides of the housing body. With this configuration, a user can rotate the adjustable curved part relative to the housing body to extend or shorten the width of the opening between the free ends of the connection curved holder and the adjustable curved holder. Accordingly, it is convenient for a user to wear or remove the respiratory filter device.

Preferably, the adjustable curved holder has a connection member formed at the connection end of the adjustable curved holder. The connection member is formed in a bowl-like shape, and has an arched peripheral sidewall extending obliquely and outward from the connection end of the adjustable curved holder; multiple elongated openings formed through the arched peripheral sidewall and extending toward a margin of the arched peripheral sidewall; and multiple arched bumps protruding from the arched peripheral sidewall and formed along the margin of the arched peripheral sidewall and between the elongated openings. The housing body of the filter module has a hole and multiple transverse elongated fins extending inward the hole and arranged along an inner wall of the hole of the housing body to form an arched surface, and the transverse elongated fins each have multiple arched holes arranged along a transverse direction of the transverse elongated fins. The shape of the arched surface corresponds to the shape of the arched peripheral sidewall, and the arched bumps of the connection member are selectively secured in a part of the arched holes of the transverse elongated fins, whereby the adjustable curved holder is rotatably connected with the housing body.

Preferably, the housing body of the filter module has an upper housing, a lower housing assembled with the upper housing, and a connection housing mounted between the upper housing and the lower housing. The upper housing has the inlet, an upper opening and an upper hole separated from each other. The lower housing has a lower opening and a lower hole separated from each other. The upper hole also faces to the lower hole to define the hole of the housing body, the upper opening faces the lower opening to define a connection opening of the housing body, and the connection housing is mounted in the connection opening.

Preferably, the connection member has a circular connection surface abutting on the connection end of the adjustable curved holder, and the hole of the housing body is larger than the circular connection surface of the connection member. Accordingly, the adjustable curved holder can be mounted in the hole of the housing body or separated from the housing body.

Preferably, the upper housing has a support and a cover pivotally connected with the support. The inlet is formed in the cover of the upper housing, the outlet is formed in the connection housing, and the support is formed with an air opening communicating with the inlet and the outlet.

Preferably, the connection curved holder and the adjustable curved holder are made of a soft material for improving the comfort of the user's skin in contact with the respiratory filter device.

Preferably, the adjustable curved holder has a connecting curved part connected with the filter module and a receiving curved part pivotally connected with the connecting curved part, the connection end of the adjustable curved holder is formed at the connecting curved part, and the free end of the adjustable curved holder is formed at the receiving curved part.

More preferably, the connecting curved part has a female lug, the receiving curved part has a male lug having a shape corresponding to a shape of the female lug, and the connecting curved part is selectively secured in a female lug of the connecting curved part.

With such a configuration, a user also can extend or shorten the width of the opening between the free ends of the connection curved holder and the adjustable curved holder by pivoting the receiving curved part. For example, a user can pivot the receiving curved part away from the free end of the connection curved holder to extend the opening between the free ends of the adjustable curved holder and the connection curved holder, such that the respiratory filter device of the present invention is easy to be worn or taken off. Also, a user can pivot the receiving curved part close to the free end of the connection curved holder and secure the male lug with the female lug to shorten the width of the opening between the free ends of the adjustable curved holder and the connection curved holder. Since the opening is smaller than the size of user's head and neck, the respiratory filter device can be securely mounted around a user's head or neck, and is beneficial for a user to do other work while wearing the respiratory filter device.

In accordance with the present invention, the female lug and the male lug are exchangeable with each other. That is, in another embodiment, the female lug is formed in a side of the receiving curved part, the male lug protrudes from a side of the connecting curved part, and the female lug and the male lug are placed at a position opposite to a pivot joint of the connecting curved part and the receiving curved part.

Preferably, the filter element is a filter film and covers the inlet of the housing body, and the filter module further has a fan mounted in the housing body and disposed between the inlet and the outlet.

Preferably, the filter module further includes a power supply unit electrically connected with the fan and a control unit electrically connected with the power supply unit.

Preferably, the filter module has a circuit board electrically connected between the fan and the power supply unit.

Preferably, the fan may be a blast fan or a centrifugal fan.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a respiratory filter device in accordance with the present invention;

FIG. 2 is an operational top view of the first embodiment of a respiratory filter device in accordance with the present invention;

FIG. 3 is a perspective view of a second embodiment of a respiratory filter device in accordance with the present invention;

FIG. 4 is an exploded perspective view of the second embodiment of a respiratory filter device in accordance with the present invention;

FIG. 5 is a side view of a support housing of the second embodiment of the respiratory filter device in accordance with the present invention;

FIG. 6 is a side view of a lower housing of the second embodiment of the respiratory filter device in accordance with the present invention;

FIG. 7 is an exploded perspective view of the second embodiment of a respiratory filter device in accordance with the present invention;

FIG. 8 is a side sectional view in partial section of the second embodiment of a respiratory filter device in accordance with the present invention;

FIG. 9 is a top sectional view in partial section of the second embodiment of a respiratory filter device in accordance with the present invention;

FIG. 10 is an operational top view of the second embodiment of a respiratory filter device in accordance with the present invention before rotating the adjustable curved holder;

FIG. 11 is another operational top view of the second embodiment of a respiratory filter device in accordance with the present invention before rotating the adjustable curved holder; and

FIG. 12 is an operational front view of the second embodiment of a respiratory filter device in accordance with the present invention, shown with the adjustable curved part separated from the housing body.

### DETAILED DESCRIPTION OF THE PREFERRED EMBODIMENTS

Hereinafter, one skilled in the arts can easily realize the advantages and effects of a respiratory filter device in accordance with the present invention from the following embodiments. The descriptions proposed herein are just preferable embodiments for the purpose of illustrations only, not intended to limit the scope of the invention. Various modifications and variations could be made in order to practice or apply the present invention without departing from the spirit and scope of the present invention.

A first embodiment of a respiratory filter device in accordance with the present invention is illustrated in FIGS. 1 and 2. The respiratory filter device comprises a filter module 10, a connection curved holder 20, an adjustable curved holder 30 and a mask 40.

The filter module 10 has a housing body 11, a filter element 12, a fan 13, a circuit board 14, a power supply unit 15, and a control unit 16.

The housing body 11 has an outlet 111 and an inlet 112 communicating with each other. The filter element 12 is mounted on the housing body 11 and is disposed between the outlet 111 and the inlet 112. In the instant embodiment, the filter element 12 is a filter film and covers the inlet 112 for filtering away the harmful substances contained in the air passing through the inlet 112 to provide purified air.

The fan 13 is mounted in the housing body 11 and between the outlet 111 and the inlet 112. The fan 13 has a rotation shaft 131 and multiple blades extending radially from the rotation shaft 131. The rotation shaft 131 has a longitudinal axis parallel to the flow direction of the inlet 112 and perpendicular to the flow direction of the outlet 111. The circuit board 14 is mounted between the fan 13 and the power supply unit 15, and is electrically connected with the fan 13 and the power supply unit 15. The power supply unit 15 is electrically connected with the fan 13 and the control unit 16.

The connection curved holder 20 is connected with the filter module 10, and has a connection end 21 and a free end 22 opposite to each other. The connection end 21 of the connection curved holder 20 is secured onto a side of the housing body 11. Further, a connection tube 23 is embedded in the connection curved holder 20 and has two opposite ends, one of the ends of the connection tube 23 is connected to the outlet 111 of the housing body 11.

The adjustable curved holder 30 is connected with the filter module 10, and has a connection end 31 and a free end 32 opposite to each other. In the instant embodiment, the connection end 31 of the adjustable curved holder 30 is secured onto another side of the housing body 11 and opposite to the connection curved holder 20. The free end 32 of the adjustable curved holder 30 faces the free end 22 of the connection curved holder 20 without contact, forming a gap between the free end 22 of the connection curved holder 20 and the free end 32 of the adjustable curved holder 30. The gap has a width W smaller than the size of a user's neck or head. The filter module 10 is connected to the connection curved holder 20 and the adjustable curved holder 30 to define an accommodation space adjacent to the gap, said accommodation space is provided for mounting the respiratory filter device around a user's neck or head.

In the instant embodiment, the adjustable curved holder 30 has a connecting curved part 33 connected with the filter module 10 and a receiving curved part 34 pivotally connected with the connecting curved part 33. The connection end 31 of the adjustable curved holder 30 is formed at the connecting curved part 33, and the free end 32 of the adjustable curved holder 30 is formed at the receiving curved part 34 apart from the connecting curved part 33. The circuit board 14 and the power supply unit 15 are mounted in the connecting curved part 33, and the control unit 16 is mounted on the connecting curved part 33 to control the operation of the fan 13.

Since the receiving curved part 34 is pivotally connected with the connecting curved part 33, the receiving curved part 34 can be rotated pivotally relative to the connecting curved part 33 and the housing body 11 to extend or shorten the width W of the gap between the free end 32 of the adjustable curved holder 30 and the free end 22 of the connection curved holder 20. Further, the connecting curved part 33 has a side and a female lug 331 formed in the side, and the receiving curved part 34 has a side abutting the side of the connecting curved part 33 and a male lug 341 protruding from the side of the connecting curved part 33. Said male lug 341 has a shape corresponding to a shape of the female lug 331. The connection curved holder 20 and the adjustable curved holder 30 are made of a soft material to improve the comfort of wearing the respiratory filter device.

The mask 40 is connected with the connection tube 23, and has a breathing space connected to the other end of the connection tube 23 and communicating with the inlet 112 of the filter module 10 by the connection tube 23. Upon covering the mask 40 over a user's mouth and nose, the ambient air from the inlet 112 passes through the filter film, and the harmful substances contained in the ambient air are filtered away by the filter film prior to inhalation, thereby ensuring that the user inhales purified air without harmful substances.

With referenced to FIGS. 1 and 2, the receiving curved part 34 is pivotally rotated relative to the connecting curved part 33 in advance to extend the width W of the gap between the free end 32 of the adjustable curved holder 30 and the free end 22 of the connection curved holder 20, which makes the respiratory filter device easy to hang around the user's head or neck. When the respiratory filter device is worn, the male lug 341 of the receiving curved part 34 is secured into the female lug 331 of the connecting curved part 33 to shorten the width W of the gap to be less than the size of the user's head or neck. Accordingly, the respiratory filter device of the present invention can be securely mounted around the user's head or neck without accidental drop.

With the arrangements that the connection curved holder 20 is connected to a side of the housing body 11 of the filter module 10 and that the connection tube 23 is embedded in the connection curved holder 20, the connection security between the connection tube 23 and the filter module 10 is ensured, thereby preventing the respiratory filter device from being dropped accidentally upon wearing.

Further, since the respiratory filter device can be worn around a user's head and neck by the connection curved holder 20 and the adjustable curved holder 30, the length of the connection tube 23 can be shortened, and the inconvenience of holding a conventional respiratory filter device in hand when doing other work can be also overcome.

A second embodiment of a respiratory filter device in accordance with the present invention is illustrated in FIG. 3. The respiratory filter device comprises a filter module 50, a connection curved holder 60, an adjustable curved holder 70 and a mask 80.

With reference to FIGS. 3 and 4, the filter module 50 has a housing body 51, a filter element 52, a fan 53, a power supply unit 54, and a control unit 55.

With reference to FIGS. 4 to 6, the housing body 51 includes an upper housing 511, a lower housing 512, and a connection housing 513. The upper housing 511 includes a support 5111 and a cover 5112 pivotally connected with the support 5111. An inlet 5141 is formed in the cover 5112, and an air opening 5142, an upper opening 5151 and an upper hole 5161 are formed in the support 5111 and separated from each other. Said inlet 5141 of the cover 5112 communicates with the air opening 5142 of the support 5111 for air flowing.

The lower housing 512 is assembled with the upper housing 511, and has a lower opening 5152 and a lower hole 5162 respectively disposed at positions at which the upper opening 5151 and the upper hole 5161 are disposed. Therefore, the upper opening 5151 and the lower opening 5152 facing to each other define a connection opening 515, and the upper hole 5161 and the lower hole 5162 facing to each other define a hole 516 for receiving the adjustable curved holder 70.

The connection housing 513 is partially mounted in the connection opening 515 and has a receiving opening 5131 and an outlet 5132 communicating with each other. In the instant embodiment, the support 5111 is connected with the cover 5112, the lower housing 512, and the connection housing 513 to compose the housing body 51, and thereby the inlet 5141 of the cover 5112 communicates with the outlet 5132 of the connection housing 513.

With reference to FIGS. 4 to 6, the housing body 51 of the filter module 50 has multiple transverse elongated fins 517, a part of the transverse elongated fins 517 are mounted on an inner wall edge of the support 5111 and extend inward the upper hole 5161, another part of the transverse elongated fins 517 are mounted on an inner wall edge of the lower housing 512 and extending inward the lower hole 5162. The transverse elongated fins 517 each have multiple arched holes 5171 arranged along a transverse direction of the transverse elongated fins 517.

With reference to FIGS. 3 and 4, the filter element 52 is mounted on the support 5111, and includes a filter film 521 and a carrier 522 surrounding the filter film 521 for holding the filter film 521. Said carrier 522 is mounted between the support 5111 and the cover 5112 of the upper housing 511, such that the filter film 521 is disposed between the inlet 5141 and the air opening 5142 to filter away and absorb the harmful substances contained in the ambient air passing through the inlet 5141.

The fan 53 is disposed in the housing body 51 and mounted between the upper housing 511, the lower housing 512 and the connection housing 513. In the instant embodiment, the fan 53 is a blast fan, and has an airflow inlet 531 and an airflow outlet 532 communicating with each other. The airflow inlet 531 of the blast fan communicates with the air opening 5142 of the support 5111 and the inlet 5141 of the cover 5112 in sequence, and the airflow outlet 532 of the blast fan communicates with the receiving opening 5131 of the connection housing 513, allowing the ambient air to flow from the environment into the breathing space of the mask 80. The fan 53 is electrically connected to the power supply unit 54. In another embodiment, the fan 53 may be a centrifugal fan.

As similar with the first embodiment, the connection curved holder 60 of the instant embodiment is connected with the filter module 50, and has a connection end 61 and a free end 62 opposite to each other. The connection end 61 is connected with a side of the housing body 51, and a connection tube 63 having two opposite ends is embedded in the connection curved holder 60. One of the ends of the connection tube 63 is connected to the outlet 5132 of the connection housing 513 to allow the connection tube 63 to communicate with the airflow outlet 532 of the blast fan through the connection housing 513.

The adjustable curved holder 70 is connected with another side of the housing body 51 and opposite to the connection curved holder 60, and has a connection end 71 and a free end 72 opposite to each other. The power supply unit 54 and the control unit 55 are mounted on the adjustable curved part 70 and are electrically connected with each other. In the instant embodiment, the free end 72 of the adjustable curved holder 70 faces the free end 62 of the connection curved holder 60 with an opening between the free ends 62, 72, and a width W of the gap is smaller than the size of a user's neck or head. The filter module 50 is connected between the connection curved holder 20 and the adjustable curved holder 30 to define an accommodation space adjacent to the gap for wearing the respiratory filter device around a user's neck or head.

More specifically, the adjustable curved holder 70 of the instant embodiment has a bowl-like connection member 73 protruding from the connection end 71 and abutting on the connection end 71 to form a circular connection surface between the overlapped connection end 71 and the connection member 73. With reference to FIGS. 4 and 7, the connection member 73 has an arched peripheral sidewall 731, multiple elongated openings 732, and multiple arched bumps 733. The arched peripheral sidewall 731 extends from the connection end 71 obliquely and outward to have an arched shape, and defines a cavity therein. The elongated openings 732 are formed through the arched peripheral sidewall 731 and extend toward a margin of the arched peripheral sidewall 731. The arched bumps 733 protrude from an outer surface of the arched peripheral sidewall 731, and the arched bumps 733 are arranged along the margin of the arched peripheral sidewall 731 and disposed between the elongated openings 732.

In accordance with the present invention, the arched bumps 733 each have a shape and a size corresponding to a shape and a size of the arched holes 5171 of the transverse elongated fins 517. Accordingly, the arched bumps 733 can be secured in the arched holes 5171 of the transverse elongated fins 517 to connect the adjustable curved holder 70 with the housing body 51.

With reference to FIGS. 8 and 9, when the support 5111 of the upper housing 511 is assembled with the lower housing 512, the transverse elongated fins 517 mounted on the support 5111 and on the lower housing 512 are arranged along an inner wall of the hole 516 to form an arched surface. Said arched surface has a shape corresponding to the arched shape of the arched peripheral sidewall 731, and the shapes of the arched bumps 733 correspond to the shapes of the arched holes 5171 of the transverse elongated fins 517. With such an arrangement, the connection member 73 can be mounted in the hole 516 of the housing body 51, and the arched bumps 733 of the connection member 73 are selectively secured in a part of the arched holes 5171, such that the adjustable curved holder 70 is connected with the housing body 51 in a rotatable connection and can be further connected with the housing body 51 at a fixed arrangement through the arched bumps 733 and the arched holes 5171 secured with each other.

In accordance with the present invention, the positions at which the arched holes 5171 and the arched bumps 733 are disposed are exchangeable with each other. For example, in another embodiment, the arched hole may be formed in the arched peripheral sidewall of the connection member, and the arched bumps may protrude on the transverse elongated fins.

With reference to FIGS. 3 and 4, the mask 80 has a breathing space 81. One of the two ends of the connection tube 63 communicates to the outlet 5132 of the connection housing 513, the airflow outlet 532, the airflow inlet 531, the air opening 5142 and the inlet 5141 in sequence, and the other end of the connection tube 63 communicates with the breathing space 81 of the mask 80. Upon covering the mask 80 over a user's mouth and nose, the breathing space 81 communicates with the environment only through the connection tube 63 and the filter module 50. Ambient air containing harmful substances can be filtered away by the filter film 521 before passing through the air opening 5142, the airflow inlet 531, the airflow outlet 532, the receiving opening 5131, the outlet 5132, and the connection tube 63 into the breathing space 81. Accordingly, the respiratory filter device can prevent a user wearing the same from directly inhaling ambient air with harmful substances.

In accordance with the present invention, the adjustable curved holder 70 may be rotated around an axis of the connection member 73, so as to adjust the width W of the gap between the free end 62 of the connection curved holder 60 and the free end 72 of the adjustable curved holder 70 and to allow a user to wear the respiratory filter device on the head or neck.

With reference to FIGS. 5 and 6, the upper hole 5161 is formed from a side of the support 5111 extending toward a front edge of the support 5111, and the upper hole 5161 formed in the support 5111 is larger than the lower hole 5162 formed in the lower housing 512. With reference to FIGS. 10 and 11, the hole 516 is larger than the circular connection surface formed between the overlapped connection end 71 and the connection member 73. With said configuration, the adjustable curved holder 70 also can be rotated toward or away from the connection curved holder 60 to shorten the width W of the gap between the free end 62 and the free end 72 (as shown in FIG. 10) or to extend the width W of the gap (as shown in FIG. 11).

Due to the technical features that the elongated openings 732 are formed through the connection member 73 and that the connection member 73 is elastic, the connection member 73 can be elastically deformed and mounted in the hole 516 of the housing body 51 by an external force, so as to connect the connection member 73 to another side of the housing body 51. A user can wear the respiratory filter device securely by hanging the connection curved holder 60 and the adjustable curved holder 70 on the user's head or neck.

With reference to FIG. 12, when a user intends to take off the respiratory filter device, the connection member 73 is compressed and deformed by an external pull force upon abutting the housing body 51, and then the adjustable curved holder 70 can be detached from the housing body 51. Accordingly, depending on need, a user can assemble or separate the adjustable curved holder 70 and the housing body 51 easily, thereby further improving the convenience upon wearing the respiratory filter device.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A respiratory filter device, having:
a filter module having a housing body and a filter element mounted on the housing body, the housing body having an inlet and an outlet communicating with the inlet, and the filter element mounted between the inlet and the outlet;
a connection curved holder connected with the filter module, and having a free end and a connection end connected with the housing body;
an adjustable curved holder connected with the filter module and opposite to the connection curved holder, the adjustable curved holder having a connection end connected with the housing body and a free end facing the free end of the connection curved holder with an gap between the free ends of the connection curved holder and the adjustable curved holder;
a connection tube embedded in the connection curved holder and having two opposite ends, one of the ends of the connection tube connected to the outlet of the housing body; and
a mask connected with the connection tube and having a breathing space communicating with the other end of the connection tube;
wherein the filter module is connected to the connection curved holder and the adjustable curved holder to define an accommodation space adjacent to the opening.

2. The respiratory filter device as claimed in claim 1, wherein the adjustable curved holder is rotatably connected with the housing body of the filter module.

3. The respiratory filter device as claimed in claim 2, wherein the adjustable curved holder has a connection member formed at the connection end of the adjustable curved holder, and the connection member has:
an arched peripheral sidewall extending obliquely and outward from the connection end of the adjustable curved holder;
multiple elongated openings formed through the arched peripheral sidewall and extending toward a margin of the arched peripheral sidewall; and
multiple arched bumps protruding from the arched peripheral sidewall and formed along the margin of the arched peripheral sidewall and between the elongated openings; and
the housing body of the filter module has:
a hole; and
multiple transverse elongated fins extending inward the hole and arranged along an inner wall of the hole of the housing body to form an arched surface, and the transverse elongated fins each having multiple arched holes arranged along a transverse direction of the transverse elongated fins;
wherein the arched bumps of the connection member are selectively secured in a part of the arched holes of the transverse elongated fins, whereby the adjustable curved holder is rotatably connected with the housing body of the filter module.

4. The respiratory filter device as claimed in claim 3, wherein the housing body of the filter module has:
an upper housing having:
the inlet;
an upper opening; and
an upper hole;
a lower housing assembled with the upper housing and having:
a lower opening facing the upper opening, and the upper opening and the lower opening defining a connection opening of the housing body; and
a lower hole facing the upper hole, and the upper hole and the lower hole defining the hole of the housing body;
a connection housing mounted between the upper housing and the lower housing and mounted in the connection opening;
wherein the filter module has a fan mounted between the upper housing and the lower housing.

5. The respiratory filter device as claimed in claim 4, wherein the connection member has a circular connection surface abutting on the connection end of the adjustable curved holder, and the hole of the housing body is larger than the circular connection surface of the connection member.

6. The respiratory filter device as claimed in claim 4, wherein the upper housing has a support and a cover pivotally connected with the support, the inlet is formed in the cover of the upper housing, the outlet is formed in the connection housing, and the support is formed with an air opening communicating with the inlet and the outlet.

7. The respiratory filter device as claimed in claim 1, wherein the adjustable curved holder has a connecting curved part connected with the filter module and a receiving curved part pivotally connected with the connecting curved part, the connection end of the adjustable curved holder is formed at the connecting curved part, and the free end of the adjustable curved holder is formed at the receiving curved part.

8. The respiratory filter device as claimed in claim 7, wherein the connecting curved part has a female lug, the receiving curved part has a male lug having a shape corresponding to a shape of the female lug, and the connecting curved part is selectively secured in the female lug of the connecting curved part.

9. The respiratory filter device as claimed in claim 8, wherein the connecting curved part and the adjustable curved part are made of a soft material.

10. The respiratory filter device as claimed in claim 1, wherein the filter element is a filter film and covers the inlet of the housing body, and the filter module has a fan mounted in the housing body and between the inlet and the outlet.

11. The respiratory filter device as claimed in claim 6, wherein the filter element is a filter film, and the filter film is formed between the inlet and the outlet.

12. The respiratory filter device as claimed in claim 8, wherein the filter element is a filter film, and the filter film is formed between the inlet and the outlet.

13. The respiratory filter device as claimed in claim 10, wherein the filter module includes a power supply unit electrically connected with the fan and a control unit electrically connected with the power supply unit.

14. The respiratory filter device as claimed in claim 11, wherein the filter module includes a power supply unit electrically connected with the fan and a control unit electrically connected with the power supply unit.

15. The respiratory filter device as claimed in claim 12, wherein the filter module includes a power supply unit electrically connected with the fan and a control unit electrically connected with the power supply unit.

16. The respiratory filter device as claimed in claim 13, wherein the fan is a blast fan.

17. The respiratory filter device as claimed in claim 13, wherein the fan is a centrifugal fan.

18. The respiratory filter device as claimed in claim 14, wherein the filter module has a circuit board electrically connected between the fan and the power supply unit.

19. The respiratory filter device as claimed in claim 15, wherein the filter module has a circuit board electrically connected between the fan and the power supply unit.
